(19) United States
(12) Patent Application Publication
(10) Pub. No.: US 2012/0277849 A1
(43) Pub. Date: Nov. 1, 2012

(54) ARTIFICIAL BLOOD VESSEL

(75) Inventors: Kazuyoshi TANI, Ashigarakami-gun (JP); Akira Sawada, Ashigarakami-gun (JP); Yoshikazu Takahashi, Ashigarakami-gun (JP)

(73) Assignee: TERUMO KABUSHIKI KAISHA, Shibuya-ku (JP)

(21) Appl. No.: 13/544,334

(22) Filed: Jul. 9, 2012

Related U.S. Application Data
(63) Continuation of application No. PCT/JP2011/053916, filed on Feb. 23, 2011.

(30) Foreign Application Priority Data
Mar. 4, 2010 (JP) 2010-047282

(51) Int. Cl. A61F 2/06

(52) U.S. Cl. 623/1.14

(57) ABSTRACT

An artificial blood vessel includes one proximal-end and at least one distal-end communicating with the proximal-end, and an inserting port positioned between the proximal-end and the distal-end. The inserting port is provided with a check valve that permits an intravascular curing device to pass through up to the proximal-end and also prevents body fluid from flowing out from the artificial blood vessel. The proximal-end is connected to the ascending aorta and the at least one distal-end is connected to one of the right brachiocephalic arterial trunk, the left common carotid artery, and the left subclavian artery.

### CROSS REFERENCES TO RELATED APPLICATIONS

This application is a continuation of International Application PCT/JP2011/053916 filed on Feb. 23, 2011, which claims priority to Japanese Patent Application No. 2010-047282 filed in the Japanese Patent Office on Mar. 4, 2010, the entire content of both of which is incorporated herein by reference.

### TECHNICAL FIELD

The present invention generally relates to an artificial blood vessel to be used to cure an aorta or the like.

### BACKGROUND DISCUSSION

A common way of curing aortic diseases, such as aortic aneurysm and aortic dissection, is by surgery involving replacement of the aortic arch with an artificial blood vessel. There have been developed various artificial blood vessels for this purpose, an example of which is disclosed in U.S. Pat. No. 6,770,090.

The surgery mentioned above requires a heart-lung machine for extracorporeal circulation. Surgery that ranges from the ascending aorta or aortic arch to the descending aorta is highly invasive, imposing a large burden on the patient, because of its extended incision.

There have been proposed various techniques to supersede the above-mentioned surgery involving replacement. One of them is intended to insert a stent graft into the aorta by partially clamping the ascending aorta, connecting an artificial blood vessel that bypasses the right brachiocephalic arterial trunk or left common carotid artery branching from the aortic arch, and finally closing the branching part of the aortic arch.

To reduce the burden and invasiveness on the patient, the foregoing technique should permit relatively easy and smooth insertion of intravascular curing devices (e.g., a delivery system for placement of a stent graft and a contrast catheter to ensure the state of placement of the stent graft) into the aorta.

### SUMMARY

Disclosed here is an artificial blood vessel that permits relatively easy and smooth insertion of intravascular curing devices, with more reduced burden and invasion on the patient.

The artificial blood vessel includes one proximal-end, at least one distal-end communicating with the proximal-end, and an inserting port positioned between the proximal-end and the distal-end, wherein the inserting port is provided with a check valve that permits an intravascular curing device to pass through up to the proximal-end and also prevents body fluid from flowing out from the artificial blood vessel.

An advantage of this construction is that the blood vessel branching from the aortic arch can be bypassed while the heart is beating, without the necessity of extracorporeal circulation through a heart-lung machine, by connecting the proximal-end to the ascending aorta, which is partially clamped, and connecting the distal-end to the blood vessel branching from the aortic arch. This leads to a reduced burden on the patient. Another advantage is that the inserting port permits relatively smooth insertion and advance of a stent graft into the aortic arch. In this way it is possible to insert an intravascular curing device, such as delivery catheter, more easily and smoothly than inserting it from the groin of the thigh.

One way of inserting a stent graft into the aortic arch, with the artificial blood vessel attached to the aorta and the blood vessel branching from the aortic arch closed, is accomplished by suturing the proximal-end of the artificial blood vessel to the ascending aorta, which is partially clamped while the heart is beating, dissecting the blood vessel, such as right brachiocephalic arterial trunk, branching from the aortic arch and suturing the distal-end of the artificial blood vessel to the dissected blood vessel, thereby closing the dissected blood vessel branching from the aortic arch, inserting a delivery catheter from the inserting port such that the delivery catheter passes through the proximal-end and advances to the aortic arch, which is the object position, thereby inserting the stent graft into the aortic arch, and removing the delivery catheter from the inserting port, followed by cutting off the inserting port and closing the cut part.

The distal-end of the artificial blood vessel may have a branched structure or three-forked structure, so that it may be sutured to more than one blood vessel. This makes the artificial blood vessel more compatible with the living body.

The inserting port mentioned above is preferably positioned between the proximal-end and the branching distal-end. This helps facilitate insertion of an intravascular curing device into the aorta or other blood vessels through the proximal-end.

The check valve mentioned above should preferably be composed of first and second valve components, the first valve component preventing body fluid from flowing out while the intravascular curing device is inserted into the inserting port, and the second valve component preventing body fluid from flowing out while the intravascular curing device is not yet inserted into the inserting port. The check valve in this structure helps ensure good sealing performance regardless of whether or not the intravascular curing device is inserted into the inserting port.

The check valve composed of two components should be constructed as follows. The second valve component is positioned closer to the front side for insertion of the intravascular curing device than the first valve component. The first valve component has a hole permitting the intravascular curing device to be smoothly inserted and to come into close contact with the external surface of the intravascular curing device which has been inserted. The second valve component has a pair of walls inclined toward the direction in which the intravascular curing device is inserted into the inserting port. Adjacent ends of the inclined walls come close each other and separate from each other so that the intravascular curing device passes through the space between the inclined walls. The thus constructed check valve is relatively simple in construction, but is capable of a relatively tight sealing regardless of whether or not the intravascular curing device is inserted into the inserting port.

The artificial blood vessel may be constructed such that the proximal-end is capable of connection to the ascending aorta and the distal-end is capable of connection to at least one of the right brachiocephalic arterial trunk, left common carotid artery, and left subclavian artery.

According to another aspect, an artificial blood vessel that is attachable to an aorta includes: a trunk tube terminating at a proximal-end configured to be sutured to the ascending aorta; a plurality of spaced apart branch tubes each fixed to the trunk tube so that the trunk tube and the branch tubes communicate with one another, with the branch tubes each terminating in a respective distal-end communicating with the proximal-end; and an inserting port fixed to the trunk portion and communicating with both the proximal-end and each of the distal-ends. The inserting port includes a check valve adapted to receive an intravascular curing device, with the check valve being configured to prevent flow of body fluid out of the inserting portion by way of the check valve when the intravascular curing device is not received in the check valve, and the check valve also being configured to prevent flow of body fluid out of the inserting portion by way of the check valve when the intravascular curing device is received in the check valve.

Another aspect involves a method of using an artificial blood vessel comprising: fixing a proximal-end of an artificial blood vessel to an ascending aorta of an aorta, the artificial blood vessel also comprising at least one distal-end communicating with the proximal-end, and an inserting port including a check valve; inserting an intravascular curing device into the inserting port; and advancing the intravascular curing device through the check valve and through the proximal-end to position the intravascular curing device in an aortic arch of the aorta while also preventing body fluid from flowing out from the artificial blood vessel.

### BRIEF DESCRIPTION OF THE DRAWINGS

FIG. 1 is a schematic diagram illustrating, by way of example, an embodiment of the artificial blood vessel disclosed here.

FIG. 2 is a perspective view of the overall (entire) structure of the artificial blood vessel disclosed here.

FIG. 3 is an exploded perspective view showing the structure of the check valve attached to the inserting port.

FIG. 4A is a longitudinal cross-sectional view (in the axial direction) of the check valve, with the intravascular curing device not inserted, and FIG. 4B is a longitudinal cross-sectional view (in the axial direction) of the check valve, with the intravascular curing device inserted.

FIG. 5 is a flow chart showing the procedure for attaching the artificial blood vessel to the aorta and then placing a stent graft at a desired position in the aorta by using the artificial blood vessel.

FIG. 6 is a diagram illustrating the artificial blood vessel, with its proximal-end connected to the ascending aorta.

FIG. 7 is a diagram illustrating the reconstruction of the blood vessels branching from the aortic arch.

FIG. 8 is a diagram illustrating the artificial blood, with the inserting port cut off, which remains attached to the aorta after insertion of a stent graft.

### DETAILED DESCRIPTION

Set forth below with reference to the accompanying drawings is a detailed description of an example of one embodiment of the artificial blood vessel disclosed here. The artificial blood vessel 10 disclosed here is used to cure a blood vessel of the living body, such as the aortic arch 12a of the aorta 12, by insertion and placement of the stent graft 13 in the aortic arch. The artificial blood vessel 10 may also be used to cure (replace) other parts of the living body, as a matter of course.

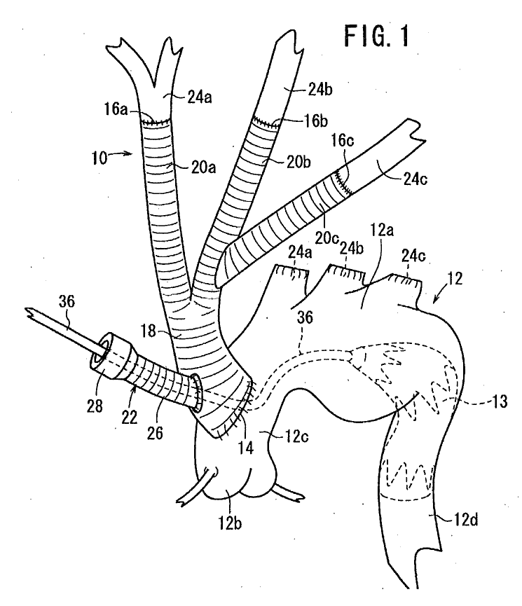

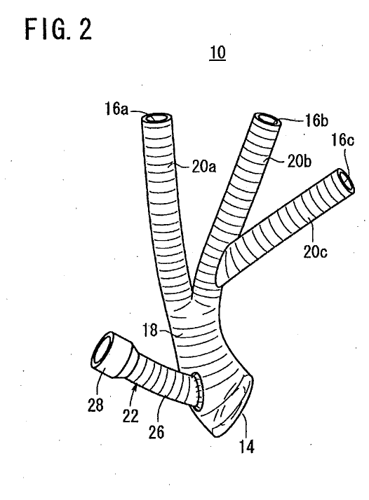

As shown in FIGS. 1 and 2, the artificial blood vessel 10 is composed of one proximal end 14 possessing a relatively larger diameter and three distal-ends 16a, 16b, 16c communicating with the proximal-end 14, the trunk tube 18 connecting the proximal-end 14 to the distal-ends 16a, 16b, the branch tubes 20a, 20b, 20c branching off from the trunk tube 18, and the inserting port 22 attached to the trunk tube 18. In this illustrated embodiment, the trunk tube 18, the branch tubes 20a, 20b, 20c and the inserting portion 22 are integrally formed in one-piece as shown in, for example, FIG. 1.

As shown in FIG. 1, the artificial blood vessel 10 is shaped with specific dimensions so that the proximal-end 14 can be connected to the ascending aorta 12c between the aortic arch 12a and the aortic valve 12b and the distal-ends 16a, 16b, 16c can be connected respectively to the right brachiocephalic arterial trunk 24a, the left common carotid artery 24b, and the left subclavian artery 24c which branch off from the aortic arch 12a. To be more specific, the trunk tube 18 has an outside diameter of about 12 mm to 30 mm, a wall thickness of about 0.1 mm to 1 mm, and a length of about 10 mm to 100 mm. Each of the branch tubes 20a, 20b, 20c has an outside diameter of about 6 mm to 10 mm, a wall thickness of about 0.1 mm to 1 mm, and a length of about 10 mm to 150 mm. The trunk tube 18 and the branch tubes 20a, 20b, 20c may be formed from the same material as used for known artificial blood vessels. Such material includes, for example, polyester fiber and ePTFE (expanded polytetrafluoroethylene).

The inserting port 22 is composed of the port tube 26 and the check valve 28. The port tube 26 is connected to that part (or the trunk tube 18 in this case) between the proximal-end 14 and each of the distal-ends 16a, 16b. The check valve 28 is attached to that part of the port tube 26 which is opposite to that part of the port tube 26 which is connected to the trunk tube 18. The port tube 26 may be formed from the same material as used for the trunk tube 18 and the branch tubes 20a and 20b. It may have an outside diameter of about 6 mm to 16 mm, a wall thickness of about 0.1 mm to 1 mm, and a length of about 10 mm to 150 mm.

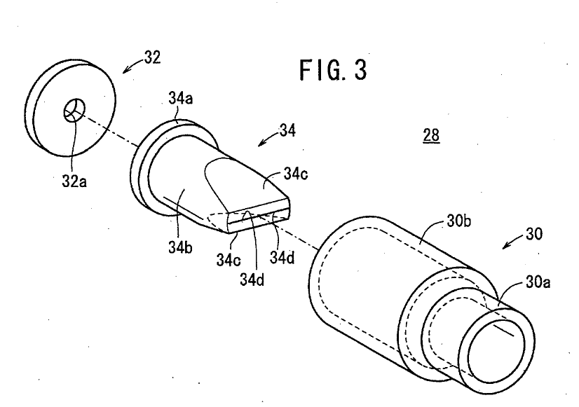

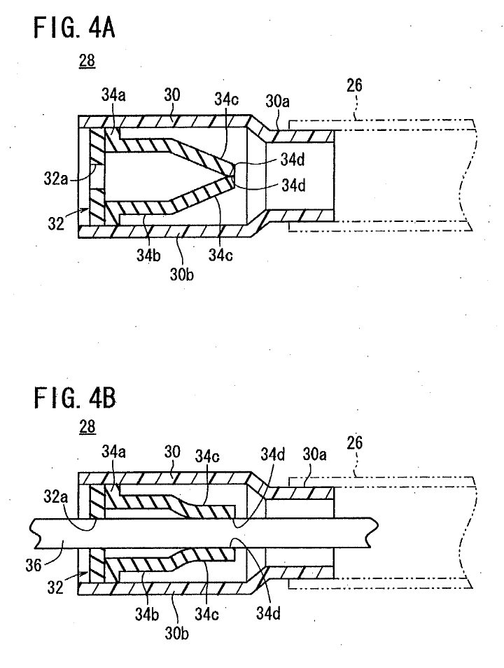

The check valve (or hemostatic valve) 28, shown in FIGS. 3, 4A, and 4B, is composed of the housing 30 and the first and second valve components 32, 34. The housing 30 is a stepped cylindrical body made of plastics material, with a smaller outer diameter portion 30a at one end fitted into the opening of the port tube 26. The first and second valve components 32, 34 are arranged in the axial direction in a larger diameter portion 30b of the interior of the housing 30 which has a larger inner and outer diameter.

The first valve component 32 is an annular-shaped discoid member having a hole 32a at its center. It is made of an elastic material such as rubber, so that it fluid-tightly fits to the inner wall of the housing 30. The hole 32a has an inside diameter large enough for the delivery catheter 36 (as an intravascular curing device) to pass through and tightly hold (tightly contact) the delivery catheter 36 passing through it. The delivery catheter 36 is intended to deliver the balloon catheter or the stent graft 13 as generally shown in FIGS. 1 and 4B.

The first valve component 32 is an ordinary rubber stopper and hence the hole 32a remains open while the delivery catheter 36 does not pass through it. Therefore, it cannot prevent body fluid (blood) from flowing out from the port tube 26 (or the aorta 12).

The second valve component 34 is arranged in the housing 30 axially adjacent to the first valve component 32 in the direction in which the delivery catheter 36 is inserted. The second valve component 34 consists of a discoid section 34a, a cylindrical section 34b, and a pair of inclined walls 34c, 34c. The discoid section 34a is fluid-tightly fixed to the inner wall of the housing 30. The cylindrical section 34b joins together the discoid section 34a and the inclined walls 34c, 34c. The inclined walls 34c, 34c are inclined toward the direction in which the delivery catheter 36 is inserted. These parts (i.e., the sections 34a, 34b and the inclined walls 34c, 34c) are formed from an elastic material such as rubber. It is also possible to configure the second valve component 34 to omit the cylindrical section 34b.

As shown in FIG. 3, the paired inclined walls 34c, 34c are formed such that their adjacent ends 34d, 34d are straight and in close contact with each other. Therefore, the paired inclined walls 34c, 34c keep the adjacent ends 34d, 34d in close contact with each other while the delivery catheter 36 is not inserted into the check valve 28, as shown in FIGS. 3 and 4A. In this state, the inclined walls 34c, 34c receive the pressure of body fluid (blood) through the port tube 26. This pressure tightly closes the adjacent ends 34d, 34d. On the other hand, when the delivery catheter 36 is inserted into the check valve 28, the adjacent ends 34d, 34d are separated from each other by the delivery catheter 36 that passes between them, as shown in FIG. 4B. In this way the delivery catheter 36 can be inserted.

The second valve component 34 is a type of valve referred to as a so-called duckbill valve. Therefore, it functions as a check valve that prevents body fluid from flowing out from the port tube 26 while the delivery catheter 36 is not inserted, as shown in FIG. 4A. However, the straight adjacent ends 34d, 34d open when the delivery catheter 36 is inserted, so that in this state the second valve component 34 hardly functions as a check valve.

As mentioned above, the check valve 28 has the first valve component 32 at its proximal-end from which the delivery catheter 36 is inserted and the second valve component 34 in front of the first valve component 32. The first valve component 32 tightly seals while the delivery catheter 36 is inserted into the inserting port 22 (or the check valve 28), and the second valve component 34 tightly seals while the delivery catheter 36 is not inserted. This structure effectively prevents body fluid from flowing out regardless of whether or not the delivery catheter 36 is inserted.

The following is a description of the technique and function in attaching the above-mentioned artificial blood vessel 10 to the aorta 12.

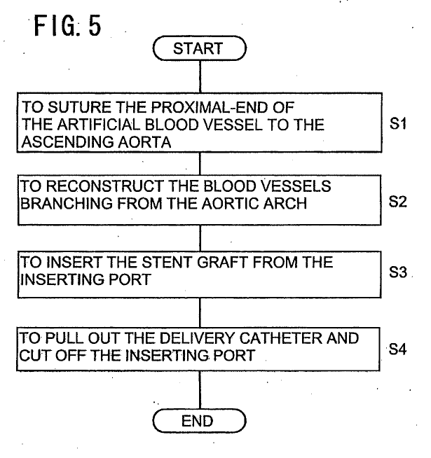

FIG. 5 is a flowchart showing a procedure for attaching the artificial blood vessel 10 to the aorta 12 and for placing the stent graft 13 at a desired position in the aorta 12 using the artificial blood vessel 10. The artificial blood vessel 10 is not intended to replace a portion of the aorta 12, but is used to bypass the right brachiocephalic arterial trunks 24a etc. (branching from the aortic arch), with the ascending aorta 12c partially clamped.

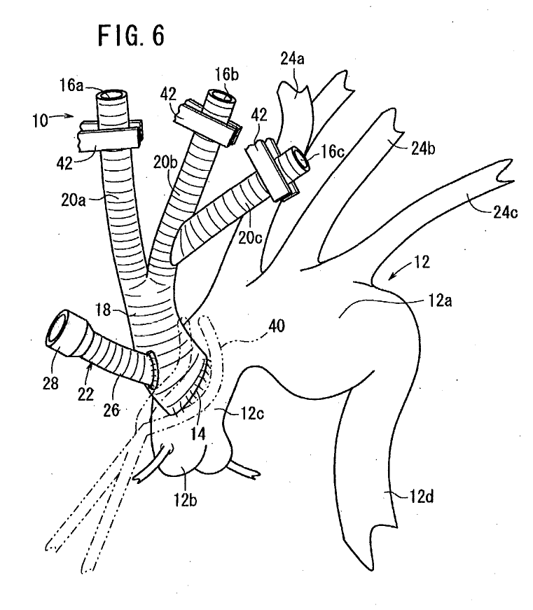

Step S1 in FIG. 5 is to suture the proximal-end 14 of the artificial blood vessel 10 to the ascending aorta 12c as shown in FIG. 6. This suturing is accomplished while partially clamping a portion of the ascending aorta 12c with a forceps 40 (while allowing the heart to beat) without extracorporeal circulation through a heart-lung machine. During Step 1, the distal-end tubes 20a, 20b, 20c are kept closed by respective forceps 42. The inserting port 22 is kept closed by the check valve 28.

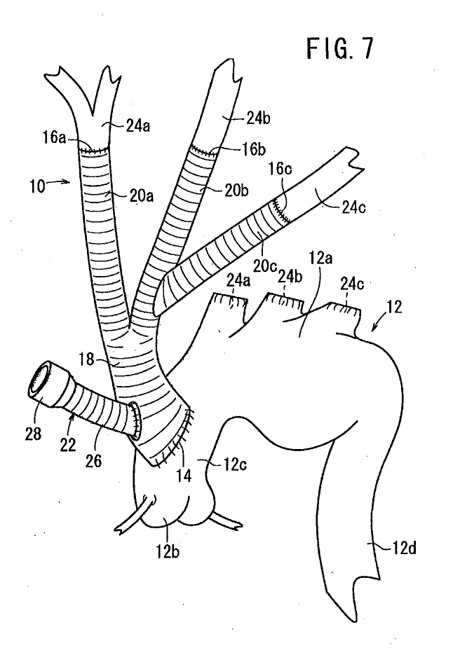

Step S2 is intended to reconstruct the blood vessels branching from the aortic arch 12a as shown in FIG. 7. That is, in this step, the right brachiocephalic arterial trunk 24a, the left common carotid artery 24b, and the left subclavian artery 24c are cut off from the aortic arch 12a, and they are respectively sutured to the distal-ends 16a, 16b, 16c of the branched tubes 20a, 20b, 20c of the artificial blood vessel 10. The dissected parts (i.e., the parts of the aortic arch 12a from which the right brachiocephalic arterial trunk 24a, the left common carotid artery 24b, and the left subclavian artery 24c are cut off) are sutured and closed.

The next step, Step S3, involves inserting the delivery catheter 36 from the inserting port 22 and advance the delivery catheter 36 through the port tube 26, the proximal-end tube 18, and the proximal-end 14 until it reaches the aortic arch 12a (the desired position), at which point the stent graft 13 is released there as shown in FIG. 1. In this way, the stent graft 13 is inserted and placed at a desired position between the aortic arch 12a and the descending aorta 12d.

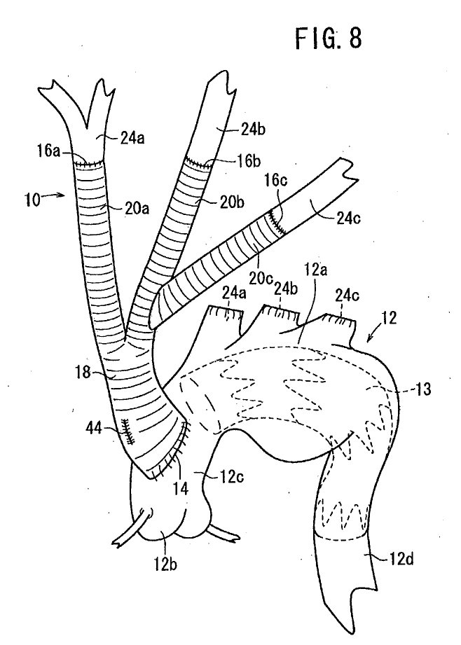

Step S4 involves pulling-out the delivery catheter 36 from the inserting port 22, cutting-off the inserting port 22 from the proximal-end tube 18, and suturing and closing the cut part 44 as shown in FIG. 8. In this way, the artificial blood vessel 10 functions as the bypass for the right brachiocephalic arterial trunk 24a, the left common carotid artery 24b, and the left subclavian artery 24c which branch off from the aortic arch 12a, and the stent graft 13 functions as a prosthesis for the aortic arch 12a.

As mentioned above, the artificial blood vessel 10 disclosed here is composed of one (only one) proximal-end 14 and three distal-ends 16a, 16b, 16c, each communicating with the proximal-end 14, and the inserting port 22 positioned between the proximal-end 14 and the distal-ends 16a, 16b, 16c, with the inserting port 22 being provided with the check valve 28.

Therefore, the artificial blood vessel 10 permits the blood vessels branching from the aortic arch to be bypassed while the heart is beating without extracorporeal circulation through a heart-lung machine as the result of connecting proximal-end 14 to the ascending aorta 12c, which is partially clamped, and the blood vessels branching from the aortic arch are connected to the distal-ends 16a, 16b, 16c. This procedure can be accomplished with less burden on the patient. Moreover, the artificial blood vessel 10 permits the stent graft 13 to be inserted and advanced to the aortic arch 12 through the inserting port 22. Thus it helps facilitate easy and smooth insertion of the intravascular curing device, such as the delivery catheter 36 (or delivery system) to place the stent graft 13 and the contrast catheter to ensure the placement of the stent graft 13. This leads to a further reduction of burden on the patient.

The artificial blood vessel 10 has at least three-forked distal-ends 16a, 16b, 16c, which facilitates connection with the branching blood vessels. In other words, a distal-end branches into at least three sections so that the distal-end is relatively easily connected to a plurality of branching blood vessels. Because of this structure, the artificial blood vessel 10 is highly compatible with the living body.

The artificial blood vessel 10 has the inserting port 22 between the distal-end (or the distal-ends 16a, 16b, 16c) and the proximal-end 14. The inserting port 22 facilitates insertion of the intravascular curing device into the blood vessel (e.g., aorta) through the proximal-end 14. Moreover, the inserting port 22 is positioned closer to the proximal-end 14 than the branched distal-ends, so that it facilitates insertion of the intravascular curing device into the aorta 12. The inserting port 22 may be positioned at the distal-end tube 20a etc. depending on the technique employed, as a matter of course.

In addition, the inserting port 22 has the check valve 28 which is composed of the first valve component 32 and the second valve component 34, as described above. The inserting port 22 of this structure relatively easily achieves high sealing performance regardless of whether or not the delivery catheter 36 is inserted into the inserting port 22, thereby inhibiting or preventing body fluid from flowing out from the inserting port 22.

The present invention is not restricted to the embodiment and variations described above, as it is possible to introduce modifications and changes in its structure and procedure within the scope of the invention. For example, the number of distal-ends 16a, 16b, 16c may be at least one or more according to the technique employed, and the number of the distal-end tubes 20a, 20b, 20c may be varied accordingly.

The check valve 28 attached to the inserting port 22 is not restricted in structure to the one consisting of the first valve component 32 and the second valve component 34 described above. It may be of any structure so long as it achieves high sealing performance regardless of whether or not the intravascular curing device is inserted into the inserting port 22.

The detailed description above describes features and aspects of one example of an embodiment of an artificial blood vessel. The present invention is not limited, however, to the precise embodiment and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. An artificial blood vessel attachable to an aorta that includes an ascending aorta, the artificial blood vessel comprising:
a trunk tube terminating at a proximal-end configured to be sutured to the ascending aorta;
a plurality of spaced apart branch tubes each fixed to the trunk tube so that the trunk tube and the branch tubes communicate with one another, the branch tubes each terminating in a respective distal-end communicating with the proximal-end;
an inserting port fixed to the trunk portion and communicating with both the proximal-end and each of the distal-ends; and
the inserting port including a check valve adapted to receive an intravascular curing device, the check valve being configured to prevent flow of body fluid out of the inserting portion by way of the check valve when the intravascular curing device is not received in the check valve, the check valve also being configured to prevent flow of body fluid out of the inserting portion by way of the check valve when the intravascular curing device is received in the check valve.

2. The artificial blood vessel according to claim 1, wherein the plurality of spaced apart branch tubes includes three spaced apart branch tubes.

3. The artificial blood vessel according to claim 1, wherein the plurality of branch tubes are connected to a distal end of the trunk tube and diverge away from one another so that the branch tubes are spaced apart from one another further distances with increasing distance away from the distal end of the branch tube.

4. The artificial blood vessel according to claim 1, wherein the inserting portion is fixed to the trunk tube at a position proximal from the distal end of the trunk tube.

5. The artificial blood vessel according to claim 1, wherein the check valve comprises a housing possessing an interior, a first valve component and a second valve component, wherein the first and second valve components are axially arranged in the interior of the housing.

6. The artificial blood vessel according to claim 1, wherein the proximal-end of the trunk tube is the only proximal end of the artificial blood vessel.

7. An artificial blood vessel comprising:
one proximal-end;
at least one distal-end communicating with the proximal-end;
an inserting port positioned between the proximal-end and the distal-end; and
the inserting port including a check valve configured to permit an intravascular curing device to pass through the check valve up to the proximal-end while also preventing body fluid from flowing out from the artificial blood vessel.

8. The artificial blood vessel according to claim 7, wherein the proximal-end is at the proximal end of a trunk tube, and the at least one distal-end is at the distal end of a branch tube that branches off from the trunk tube.

9. The artificial blood vessel according to claim 7, wherein the at least one distal-end comprises three separate distal-ends each positioned at a distal end of a respective one of three branch tubes, the three branch tubes branching away from each other.

10. The artificial blood vessel according to claim 9, wherein the inserting port is positioned between a proximal end of the three branch tubes and the proximal-end.

11. The artificial blood vessel according to claim 7, wherein the check valve includes a first valve component that prevents body fluid from flowing out when the intravascular curing device is located in the inserting port and passing through the check valve, the check valve also including a second valve component that prevents body fluid from flowing out when the intravascular curing device is not located in the inserting port and is not passing through the check valve.

12. The artificial blood vessel according to claim 11, wherein:
the first valve component is positioned closer to an open distal end of the insertion portion than the second valve component,
the first valve component includes a hole in which the intravascular curing device is slidably positionable,
the second valve component includes a pair of walls inclined towards each other in a direction away from the first valve component, adjacent ends of the inclined walls approaching each other and being separable from each other when the intravascular curing device passes through a space between the inclined walls.

13. The artificial blood vessel according to claim 7, wherein the proximal-end is connectable to an ascending aorta, and the distal-end is connectable to at least one of a right brachiocephalic arterial trunk, a the left common carotid artery, and a left subclavian artery.

14. A method of using an artificial blood vessel comprising:
fixing a proximal-end of an artificial blood vessel to an ascending aorta of a heart of a living body, the artificial blood vessel also comprising at least one distal-end communicating with the proximal-end, and an inserting port including a valve configured to prevent bodily fluid flow through the inserting port;
inserting an intravascular curing device into the inserting port;
advancing the intravascular curing device so that the intravascular curing device passes through the valve; and
further advancing the intravascular curing device through the proximal-end to position the intravascular curing device in an aortic arch of the heart while also preventing body fluid from flowing out of the inserting port by virtue of the valve.

15. The method according to claim 14, further comprising cutting-off at least one of a right brachiocephalic arterial trunk, a left common carotid artery, and a left subclavian artery from the aortic arch, and suturing at least one of the right brachiocephalic arterial trunk, the left common carotid artery, and the left subclavian artery to the distal-end of the artificial blood vessel.

16. The method according to claim 14, wherein the artificial blood vessel comprises three spaced apart distal-ends, and further comprising:
cutting-off a right brachiocephalic arterial trunk, a left common carotid artery, and a left subclavian artery from the aortic arch; and
suturing the right brachiocephalic arterial trunk to a first one of the three distal-ends of the artificial blood vessel, suturing the left common carotid artery to a second one of the three distal-ends of the artificial blood vessel, and suturing the left subclavian artery to a third one of the distal-ends of the artificial blood vessel.

17. The method according to claim 14, wherein the intravascular curing device is advanced through the valve and through the proximal-end by way of a delivery catheter, the delivery catheter being removed through the inserting portion after positioning the intravascular curing device in the aortic arch.

18. The method according to claim 14, wherein the fixing of the proximal-end of the artificial blood vessel to the ascending aorta comprises suturing the proximal-end of the artificial blood vessel to the ascending aorta.

19. The method according to claim 14, wherein the fixing of the proximal-end of the artificial blood vessel to the ascending aorta is performed while clamping a portion of the ascending aorta and while allowing the heart to beat.
